(19) United States
(12) Patent Application Publication
(10) Pub. No.: US 2008/0227786 A1
(43) Pub. Date: Sep. 18, 2008

(54) Novel Crystalline Salts of a Dipeptidyl Peptidase-IV Inhibitor

(76) Inventors: Russell R. Ferlita, Westfield, NJ (US); Karl Hansen, Atlantic Highlands, NJ (US); Vicky K. Vydra, Fair Lawn, NJ (US); Yaling Wang, Westfield, NJ (US); Christopher M. Lindemann, Loveland, CO (US)

Correspondence Address: MERCK AND CO., INC, P O BOX 2000, RAHWAY, NJ, 07065-0907, US

(21) Appl. No.: 10/585,603

(22) PCT Filed: Jan. 12, 2005
(86) PCT No.: PCT/US2005/000951

§ 371 Date: Jul. 11, 2006

Related U.S. Application Data
(60) Provisional application No. 60/537,073, filed on Jan. 16, 2004.

(51) Int. Cl. A61K 31/4985; C07D 487/02; A61P 3/10

(52) U.S. Cl. 514/249; 544/350

(57) ABSTRACT

Novel crystalline salts of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-&agr; amine are potent inhibitors of dipeptidyl peptidase-IV and are useful for the treatment of non-insulin dependent (Type 2) diabetes mellitus. The invention also relates to pharmaceutical compositions containing these novel salts, processes to prepare these salts and their pharmaceutical compositions as well as uses thereof for the treatment of Type 2 diabetes.

### FIELD OF THE INVENTION

The present invention relates to novel crystalline salts of a dipeptidyl peptidase-IV inhibitor. More particularly, the invention relates to novel crystalline hydrochloric acid, benzenesulfonic acid, p-toluenesulfonic acid, 10-camphorsulfonic acid, and tartaric acid salts of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, which is a potent inhibitor of dipeptidyl peptidase-IV. These novel crystalline salts, and hydrates thereof, are useful for the treatment and prevention of diseases and conditions for which an inhibitor of dipeptidyl peptidase-IV is indicated, in particular Type 2 diabetes, obesity, and high blood pressure. The invention further concerns pharmaceutical compositions comprising the novel crystalline salts of the present invention, or hydrates thereof, useful to treat Type 2 diabetes, obesity, and high blood pressure as well as processes for the preparation of such salts and their pharmaceutical compositions.

### BACKGROUND OF THE INVENTION

Inhibition of dipeptidyl peptidase-IV (DPP-IV), an enzyme that inactivates both glucose-dependent insulinotropic peptide (GIP) and glucagon-like peptide 1 (GLP-1), represents a novel approach to the treatment and prevention of Type 2 diabetes, also known as non-insulin dependent diabetes mellitus (NIDDM). The therapeutic potential of DPP-IV inhibitors for the treatment of Type 2 diabetes has been reviewed: C. F. Deacon and J. J. Holst, “Dipeptidyl peptidase IV inhibition as an approach to the treatment and prevention of Type 2 diabetes: a historical perspective,” Biochem. Biophys. Res. Commun., 294: 1-4 (2000); K. Augustyns, et al., “Dipeptidyl peptidase IV inhibitors as new therapeutic agents for the treatment of Type 2 diabetes,” Expert. Opin. Ther. Patents, 13: 499-510 (2003); D. J. Drucker, “Therapeutic potential of dipeptidyl peptidase IV inhibitors for the treatment of Type 2 diabetes,” Expert Opin. Investig. Drugs, 12: 87-100 (2003); and M. A. Nauck et al., “Incretins and Their Analogues as New Antidiabetic Drugs,” Drug News Perspect., 16: 413-422 (2003).

U.S. Pat. No. 6,699,871 (issued Mar. 2, 2004) and WO 03/004498 (published 16 Jan. 2003), both assigned to Merck & Co., describe a class of beta-amino tetrahydrotriazolo-[4,3-α]pyrazines, which are potent inhibitors of DPP-IV and therefore useful for the treatment of Type 2 diabetes. Specifically disclosed in U.S. Pat. No. 6,699,871 and WO 03/004498 is (2R)-4-oxo-4-[3-(trifluoromethyl)-5,6-dihydro[1,2,4]-triazolo[4,3-α]pyrazin-7(8H)-yl]-1-(2,4,5-trifluorophenyl)butan-2-amine. Pharmaceutically acceptable salts of this compound are generically encompassed within the scope of WO 03/004498 and U.S. Pat. No. 6,699,871.

However, there is no specific disclosure in WO 03/004498 and U.S. Pat. No. 6,699,871 of the newly discovered crystalline hydrochloric acid, benzenesulfonic acid, p-toluenesulfonic acid, 10-camphorsulfonic acid, or tartaric acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I below.

### SUMMARY OF THE INVENTION

The present invention is concerned with novel crystalline hydrochloric acid, benzenesulfonic acid, p-toluenesulfonic acid, 10-camphorsulfonic acid, and tartaric acid salts of the dipeptidyl peptidase-IV (DPP-IV) inhibitor (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine. Such salts, and hydrates thereof, have advantages in the preparation of pharmaceutical compositions of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine, such as ease of processing, handling, and dosing. In particular, they exhibit improved physicochemical properties, such as solubility, stability to stress, and rate of solution, rendering them particularly suitable for the manufacture of various pharmaceutical dosage forms. The invention also concerns pharmaceutical compositions containing the novel salts, or hydrates thereof, as well as methods for using them as DPP-IV inhibitors, in particular for the prevention or treatment of Type 2 diabetes, obesity, and high blood pressure.

### BRIEF DESCRIPTION OF THE FIGURES

FIG. 1 is a characteristic X-ray diffraction pattern of the crystalline hydrochloric acid salt monohydrate of Compound I of the present invention.

FIG. 2 is a typical thermogravimetric analysis (TGA) curve of the crystalline hydrochloric acid salt monohydrate of Compound I of the present invention.

FIG. 3 is a typical differential scanning calorimetry (DSC) curve of the crystalline hydrochloric acid salt monohydrate of Compound I of the present invention.

FIG. 4 is a characteristic X-ray diffraction pattern of the crystalline L-tartaric acid salt hemihydrate of Compound I of the present invention.

FIG. 5 is a typical thermogravimetric analysis (TGA) curve of the crystalline L-tartaric acid salt hemihydrate of Compound I of the present invention.

FIG. 6 is a typical differential scanning calorimetry (DSC) curve of the crystalline L-tartaric acid salt hemihydrate of Compound I of the present invention.

FIG. 7 is a characteristic X-ray diffraction pattern of the crystalline benzenesulfonic acid salt anhydrate of Compound I of the present invention.

FIG. 8 is a typical thermogravimetric analysis (TGA) curve of the crystalline benzenesulfonic acid salt anhydrate of Compound I of the present invention.

FIG. 9 is a typical differential scanning calorimetry (DSC) curve of the crystalline benzenesulfonic acid salt anhydrate of Compound I of the present invention.

FIG. 10 is a characteristic X-ray diffraction pattern of the crystalline p-toluenesulfonic acid salt anhydrate of Compound I of the present invention.

FIG. 11 is a typical thermogravimetric analysis (TGA) curve of the crystalline p-toluenesulfonic acid salt anhydrate of Compound I of the present invention.

FIG. 12 is a typical differential scanning calorimetry (DSC) curve of the crystalline p-toluenesulfonic acid salt anhydrate of Compound I of the present invention.

FIG. 13 is a characteristic X-ray diffraction pattern of the crystalline (1S)-(+)-10-camphorsulfonic acid salt anhydrate of Compound I of the present invention.

FIG. 14 is a typical thermogravimetric analysis (TGA) curve of the crystalline (1S)-(+)-10-camphorsulfonic acid salt anhydrate of Compound I of the present invention.

FIG. 15 is a typical differential scanning calorimetry (DSC) curve of the crystalline (1S)-(+)-10-camphorsulfonic salt anhydrate of Compound I of the present invention.

### DETAILED DESCRIPTION OF THE INVENTION

This invention provides a crystalline acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I (Compound I):

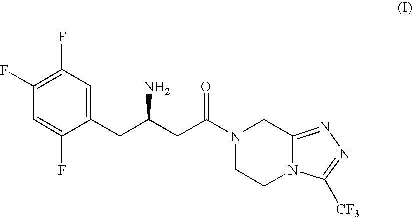

or a hydrate thereof;
wherein the acid is selected from the group consisting of hydrochloric acid, tartaric acid, benzenesulfonic acid, p-toluenesulfonic acid, and 10-camphorsulfonic acid.

One embodiment of the present invention provides a crystalline hydrochloric acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (Compound I).

In a class of this first embodiment the crystalline hydrochloric acid salt of Compound I is in the form of a monohydrate.

A second embodiment of the present invention provides a crystalline tartaric acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (Compound I).

In a class of this second embodiment the crystalline tartaric acid salt is the crystalline L-tartaric acid salt. In a second class of this embodiment the crystalline tartaric acid salt is the crystalline D-tartaric acid salt. In a third class the crystalline tartaric acid salt is the crystalline racemic DL tartaric acid salt. In a subclass of this third class, the crystalline tartaric acid salt of Compound I is in the form of a hemihydrate.

A third embodiment of the present invention provides a crystalline benzenesulfonic acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (Compound I).

In a class of this third embodiment the crystalline benzenesulfonic acid salt of Compound I is in the form of an anhydrate.

A fourth embodiment of the present invention provides a crystalline p-toluenesulfonic acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (Compound I).

In a class of this fourth embodiment the crystalline p-toluenesulfonic acid salt of Compound I is in the form of an anhydrate.

A fifth embodiment of the present invention provides a crystalline 10-camphorsulfonic acid salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (Compound I).

In a class of this fifth embodiment the crystalline 10-camphorsulfonic salt is the crystalline (1R)-(−)-camphorsulfonic acid salt. In a second class the crystalline 10-camphorsulfonic salt is the crystalline (1S)-(+)-camphorsulfonic acid salt. In a third class the crystalline 10-camphorsulfonic acid salt is the crystalline racemic (±)-10-camphorsulfonic acid salt. In a subclass of this third class, the crystalline 10-camphorsulfonic acid salt of compound I is in the form of an anhydrate.

A further embodiment of the present invention provides a particular salt drug substance that comprises a crystalline salt of the present invention present in a detectable amount. By “drug substance” is meant the active pharmaceutical ingredient. The amount of crystalline salt in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. In a class of this embodiment, about 5% to about 100% by weight of the crystalline salt of the present invention is present in the drug substance. In a second class of this embodiment, about 10% to about 100% by weight of the crystalline salt is present in the drug substance. In a third class of this embodiment, about 25% to about 100% by weight of the crystalline salt is present in the drug substance. In a fourth class of this embodiment, about 50% to about 100% by weight of the crystalline salt is present in the drug substance. In a fifth class of this embodiment, about 75% to about 100% by weight of the crystalline salt is present in the drug substance. In a sixth class of this embodiment, substantially all of the salt drug substance is the crystalline salt of the present invention, i.e., the salt drug substance is substantially phase pure crystalline salt.

The crystalline salts of the present invention exhibit pharmaceutic advantages over the free base and the previously disclosed amorphous hydrochloric acid salt (WO 03/004498) in the preparation of a pharmaceutical drug product containing the pharmacologically active ingredient. In particular, the enhanced chemical and physical stability of the crystalline salts constitute advantageous properties in the preparation of solid pharmaceutical dosage forms containing the pharmacologically active ingredient.

The crystalline salts of the present invention, which exhibit potent DPP-IV inhibitory properties, are particularly useful for the prevention or treatment of Type 2 diabetes, obesity, and high blood pressure.

Another aspect of the present invention provides a method for the prevention or treatment of clinical conditions for which an inhibitor of DPP-IV is indicated, which method comprises administering to a patient in need of such prevention or treatment a prophylactically or therapeutically effective amount of a crystalline salt of the present invention, or a hydrate thereof. Such clinical conditions include diabetes, in particular Type 2 diabetes, hyperglycemia, insulin resistance, and obesity.

The present invention also provides for the use of a crystalline salt of Compound I of the present invention, or a hydrate thereof, for the prevention or treatment in a mammal of clinical conditions for which an inhibitor of DPP-IV is indicated, in particular Type 2 diabetes, hyperglycemia, insulin resistance, and obesity.

The present invention also provides for the use of a crystalline salt of Compound I of the present invention, or a hydrate thereof, for the manufacture of a medicament for the prevention or treatment in a mammal of clinical conditions for which an inhibitor of DPP-IV is indicated, in particular Type 2 diabetes, hyperglycemia, insulin resistance, and obesity.

The present invention also provides pharmaceutical compositions comprising a crystalline salt of the present invention, or a hydrate thereof, in association with one or more pharmaceutically acceptable carriers or excipients. In one embodiment the pharmaceutical composition comprises a therapeutically effective amount of the active pharmaceutical ingredient in admixture with pharmaceutically acceptable excipients wherein the active pharmaceutical ingredient comprises a detectable amount of a crystalline salt of the present invention. In a second embodiment the pharmaceutical composition comprises a therapeutically effective amount of the active pharmaceutical ingredient in admixture with pharmaceutically acceptable excipients wherein the active pharmaceutical ingredient comprises about 5% to about 100% by weight of a crystalline salt of the present invention. In a class of this second embodiment, the active pharmaceutical ingredient in such compositions comprises about 10% to about 100% by weight of the crystalline salt. In a second class of this embodiment, the active pharmaceutical ingredient in such compositions comprises about 25% to about 100% by weight of the crystalline salt. In a third class of this embodiment, the active pharmaceutical ingredient in such compositions comprises about 50% to about 100% by weight of the crystalline salt. In a fourth class of this embodiment, the active pharmaceutical ingredient in such compositions comprises about 75% to about 100% by weight of the crystalline salt. In a fifth class of this embodiment, substantially all of the active pharmaceutical ingredient is the crystalline salt of the present invention, i.e., the active pharmaceutical ingredient is substantially phase pure crystalline salt.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 200 mg of active ingredient. Intravenously, the most preferred doses will range from about 0.1 to about 10 mg/kg/minute during a constant rate infusion. Advantageously, the crystalline salts of the present invention may be administered in a single daily dose, or the total daily dosage may be administered in divided doses of two, three or four times daily. Furthermore, the crystalline salts of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the crystalline salts and their hydrates herein described in detail can form the active pharmaceutical ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The crystalline salts of Compound I of the present invention have been found to possess a high solubility in water, rendering them especially amenable to the preparation of formulations, in particular intranasal and intravenous formulations, which require relatively concentrated aqueous solutions of active pharmaceutical ingredient.

In a still further aspect, the present invention provides a method for the treatment and/or prevention of clinical conditions for which a DPP-IV inhibitor is indicated, which method comprises administering to a patient in need of such prevention or treatment a prophylactically or therapeutically effective amount of a crystalline salt of Compound I as defined above or a hydrate thereof in combination with another agent useful for the treatment of Type 2 diabetes, obesity, and high blood pressure.

Compounds described herein may exist as tautomers such as keto-enol tautomers. The individual tautomers as well as mixtures thereof are encompassed with compounds of structural formula I.

The term ∂% enantiomeric excess” (abbreviated “ee”) shall mean the % major enantiomer less the % minor enantiomer. Thus, a 70% enantiomeric excess corresponds to formation of 85% of one enantiomer and 15% of the other. The term “enantiomeric excess” is synonymous with the term “optical purity.”

According to a further aspect, the present invention provides a process for the preparation of the crystalline salts of Compound I of the present invention, which process comprises treating a solution of free base (2R)-4-oxo-4-[3-(trifluoromethyl)-5,6-dihydro[1,2,4]-triazolo[4,3-α]pyrazin-7(8H)-yl]-1-(2,4,5-trifluorophenyl)butan-2-amine (Compound I):

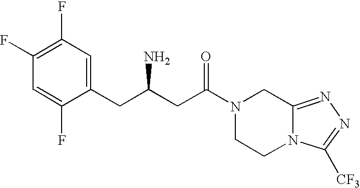

in a suitable organic solvent with a solution of the appropriate acid in a suitable organic solvent or water or mixture thereof. The process is carried out generally at about 0° C. to about 100° C., and preferably at about 20° C. to about 60° C. Generally, the organic solvent is a linear or branched C₁₋₄ alkanol, such as methanol, ethanol, or isopropanol (EPA), a linear or branched C₁₋₄ alkyl acetate, such as ethyl acetate or isopropyl acetate, diethyl ether, tetrahydrofuran, toluene, acetone, or acetonitrile. A mixture of water and the organic solvent may also be employed. Crystallization is then effected by cooling the mixture and optional seeding with crystals of the authentic acid salt, but the latter is not essential. The acid salts are then isolated by filtration and drying.

Compound I can be prepared by the procedures detailed in Schemes 1 and 2 below.

### Preparation of 3-(trifluoromethyl)-5,6,7,8-tetrahydro[1,2,4]triazolo[4,3-a]pyrazine hydrochloric acid (1-4)

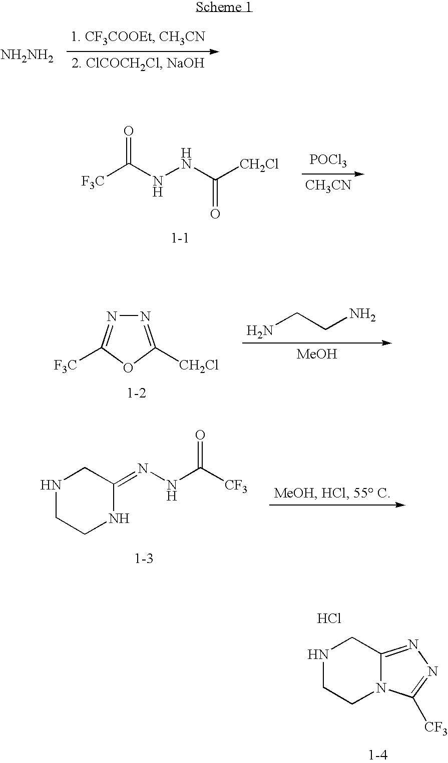

### Step A: Preparation of bishydrazide (1-1)

Hydrazine (20.1 g, 35 wt % in water, 0.22 mol) was mixed with 310 mL of acetonitrile. 31.5 g of ethyl trifluoroacetate (0.22 mol) was added over 60 min. The internal temperature was increased to 25° C. from 14° C. The resulting solution was aged at 22-25° C. for 60 min. The solution was cooled to 7° C. 17.9 g of 50 wt % aqueous NaOH (0.22 mol) and 25.3 g of chloroacetyl chloride (0.22 mol) were added simultaneously over 130 min at a temperature below 16° C. When the reaction was complete, the mixture was vacuum distilled to remove water and ethanol at 27˜30° C. and under 26˜27 in Hg vacuum. During the distillation, 720 mL of acetonitrile was added slowly to maintain constant volume (approximately 500 ml). The slurry was filtered to remove sodium chloride. The cake was rinsed with about 100 mL of acetonitrile. Removal of the solvent afforded bis-hydrazide 1-1 (43.2 g, 96.5% yield, 94.4 area % pure by HPLC assay).

¹H-NMR (400 MHz, DMSO-d6): δ 4.2 (s, 2H), 10.7 (s, 1H), and 11.6 (s, 1H) ppm.

¹³C-NMR (100 MHz, DMSO-d6): δ 41.0, 116.1 (q, J=362 Hz), 155.8 (q, J=50 Hz), and 165.4 ppm.

### Step B: Preparation of 5-(trifluoromethyl)-2-(chloromethyl)-1,3,4-oxadiazole (1-2)

Bishydrazide 1-1 from Step A (43.2 g, 0.21 mol) in ACN (82 mL) was cooled to 5° C. Phosphorus oxychloride (32.2 g, 0.21 mol) was added, maintaining the temperature below 10° C. The mixture was heated to 80° C. and aged at this temperature for 24 h until HPLC showed less than 2 area % of 1-1. In a separate vessel, 260 mL of IPAc and 250 mL of water were mixed and cooled to 0° C. The reaction slurry was charged to the quench keeping the internal temperature below 10° C. After the addition, the mixture was agitated vigorously for 30 min, the temperature was increased to room temperature and the aqueous layer was cut. The organic layer was then washed with 215 mL of water, 215 mL of 5 wt % aqueous sodium bicarbonate and finally 215 mL of 20 wt % aqueous brine solution. HPLC assay yield after work up was 86-92%. Volatiles were removed by distillation at 75-80 mm Hg, 55° C. to afford an oil which could be used directly in Step C without further purification. Otherwise the product can be purified by distillation to afford 1-2 in 70-80% yield.

¹H-NMR (400 MHz, CDCl3): δ 4.8 (s, 2H) ppm.

¹³C-NMR (100 MHz, CDCl3): δ 32.1, 115.8 (q, J=337 Hz), 156.2 (q, J=50 Hz), and 164.4 ppm.

### Step C: Preparation of N-[(2Z)-piperazin-2-ylidene]trifluoroacetohydrazide (1-3)

To a solution of ethylenediamine (33.1 g, 0.55 mol) in methanol (150 mL) cooled at −20° C. was added distilled oxadiazole 1-2 from Step B (29.8 g, 0.16 mol) while keeping the internal temperature at −20° C. After the addition was complete, the resulting slurry was aged at −20° C for 1 h. Ethanol (225 mL) was then charged and the slurry slowly warmed to −5° C. After 60 min at −5° C., the slurry was filtered and washed with ethanol (60 mL) at −5° C. Amidine 1-3 was obtained as a white solid in 72% yield (24.4 g, 99.5 area wt % pure by HPLC).

¹H-NMR (400 MHz, DMSO-d₆): δ 2.9 (t, 2H), 3.2 (t, 2H), 3.6 (s, 2H), and 8.3 (b, 1H) ppm.

¹³C-NMR (100 MHz, DMSO-d₆): δ 40.8, 42.0, 43.3, 119.3 (q, J=350 Hz), 154.2, and 156.2 (q, J=38 Hz) ppm.

### Step D: Preparation of 3-(trifluoromethyl)-5,6,7,8-tetrahydro[1,2,4]triazolo[4,3-α]pyrazine hydrochloric acid (1-4)

A suspension of amidine 1-3 (27.3 g, 0.13 mol) in 110 mL of methanol was warmed to 55° C. 37% Hydrochloric acid (11.2 mL, 0.14 mol) was added over 15 min at this temperature. During the addition, all solids dissolved resulting in a clear solution. The reaction was aged for 30 min. The solution was cooled down to 20° C. and aged at this temperature until a seed bed formed (10 min to 1 h). 300 mL of MTBE was charged at 20° C. over 1 h. The resulting slurry was cooled to 2° C., aged for 30 min and filtered. Solids were washed with 50 mL of ethanol:MTBE (1:3) and dried under vacuum at 45° C. Yield of triazole 1-4 was 26.7 g (99.5 area wt % pure by HPLC).

¹H-NMR (400 MHz, DMSO-d₆): δ 3.6 (t, 2H), 4.4 (t, 2H), 4.6 (s, 2H), and 10.6 (b, 2H) ppm;

¹³C-NMR (100 MHz, DMSO-d₆): δ 39.4, 39.6, 41.0, 118.6 (q, J =325 Hz), 142.9 (q, J=50 Hz), and 148.8 ppm.

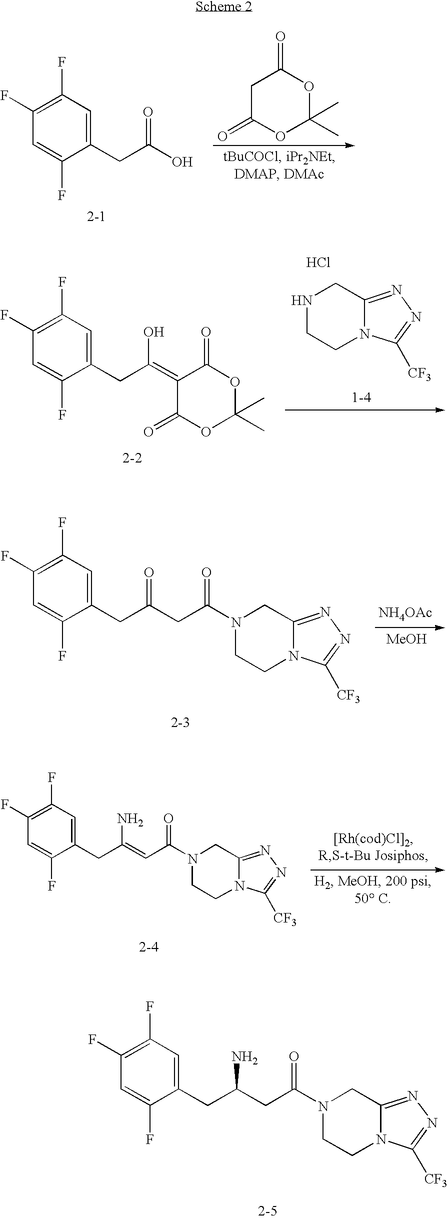

### Step A: Preparation of 4-oxo-4-[3-(trifluoromethyl)-5,6-dihydro[1,2,4]triazolo[4,3-α]pyrazin-7(8H)-yl]-1-(2,4,5-trifluorophenyl)butan-2-one (2-3)

2,4,5-Trifluorophenylacetic acid (2-1) (150 g, 0.789 mol), Meldrum's acid (125 g, 0.868 mol), and 4-(dimethylamino)pyridine (DMAP) (7.7 g, 0063 mol) were charged into a 5 L three-neck flask. N,N-Dimethylacetamide (DMAc) (525 mL) was added in one portion at room temperature to dissolve the solids. N,N-diisopropylethylamine (282 mL, 1.62 mol) was added in one portion at room temperature while maintaining the temperature below 40° C. Pivaloyl chloride (107 mL, 0.868 mol) was added dropwise over 1 to 2 h while maintaining the temperature between 0 and 5° C. The reaction mixture was aged at 5° C. for 1 h. Triazole hydrochloric acid 1-4 (180 g, 0.789 mol) was added in one portion at 40-50° C. The reaction solution was aged at 70° C. for several h. 5% Aqueous sodium hydrogencarbonate solution (625 mL) was then added dropwise at 20-45° C. The batch was seeded and aged at 20-30° C. for 1-2 h. Then an additional 525 mL of 5% aqueous sodium hydrogencarbonate solution was added dropwise over 2-3 h. After aging several h at room temperature, the slurry was cooled to 0-5° C. and aged 1 h before filtering the solid. The wet cake was displacement-washed with 20% aqueous DMAc (300 mL), followed by an additional two batches of 20% aqueous DMAc (400 mL), and finally water (400 mL). The cake was suction-dried at room temperature. The isolated yield of final product 2-3 was 89%.

### Step B: Preparation of (2Z)-4-oxo-4-[3-(trifluoromethyl)-5,6-dihydro[1,2,4]triazolo[4,3-α]pyrazin-7(8H)-yl]-1-(2,4,5-trifluorophenyl)but-2-en-2-amine (2-4)

A 5 L round-bottom flask was charged with methanol (100 mL), the ketoamide 2-3 (200 g), and ammonium acetate (110.4 g). Methanol (180 mL) and 28% aqueous ammonium hydroxide (58.6 mL) were then added keeping the temperature below 30° C. during the addition. Additional methanol (100 mL) was added to the reaction mixture. The mixture was heated at reflux temperature and aged for 2 h. The reaction was cooled to room temperature and then to about 5° C. in an ice-bath. After 30 min, the solid was filtered and dried to afford 2-4 as a solid (180 g); m.p. 271.2° C.

### Step C: Preparation of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (2-5)

Into a 500 ml flask were charged chloro(1,5-cyclooctadiene)rhodium(I) dimer {[Rh(cod)Cl]₂)(292 mg, 1.18 mmol) and (R,S) t-butyl Josiphos (708 mg, 1.3 mmol) under a nitrogen atmosphere. Degassed MeOH was then added (200 mL) and the mixture was stirred at room temperature for 1 h. Into a 4 L hydrogenator was charged the enamine amide 2-4 (118 g, 0.29 mol) along with MeOH (1 L). The slurry was degassed. The catalyst solution was then transferred to the hydrogenator under nitrogen. After degassing three times, the enamine amide was hydrogenated under 200 psi hydrogen gas at 50° C. for 13 h. Assay yield was determined by HPLC to be 93% and optical purity to be 94% ee.

The optical purity was further enhanced in the following manner. The methanol solution from the hydrogenation reaction (18 g in 180 mL MeOH) was concentrated and switched to methyl t-butyl ether (MTBE) (45 mL). Into this solution was added aqueous H₃PO₄ solution (0.5 M, 95 mL). After separation of the layers, 3N NaOH (35 mL) was added to the water layer, which was then extracted with MTBE (180 mL+100 mL). The MTBE solution was concentrated and solvent switched to hot toluene (180 mL, about 75° C.). The hot toluene solution was then allowed to cool to 0° C. slowly (5-10 h). The crystals were isolated by filtration (13 g, yield 72%, 98-99% ee); m.p. 114.1-115.7° C.

¹H NMR (300 Mz, CD₃CN): δ 7.26 (m), 7.08 (m), 4.90 (s), 4.89 (s), 4.14 (m), 3.95 (m), 3.40 (m), 2.68 (m), 2.49 (m), 1.40 (bs).

Compound 2-5 exists as amide bond rotamers. Unless indicated, the major and minor rotamers are grouped together since the carbon-13 signals are not well resolved:

¹³C NMR (CD₃CN): δ 171.8, 157.4 (ddd, J_{CF}=242.4, 9.2, 2.5 Hz), 152.2 (major), 151.8 (minor), 149.3 (ddd; J_{CF}=246.7, 14.2, 12.9 Hz), 147.4 (ddd, J_{CF}=241.2, 12.3, 3.7 Hz), 144.2 (q, J_{CF}=38.8 Hz), 124.6 (ddd, J_{CF}=18.5, 5.9, 4.0 Hz), 120.4 (dd, J_{CF}=19.1, 6.2 Hz), 119.8 (q, J_{CF}=268.9 Hz), 106.2 (dd, J_{CF}=29.5, 20.9 Hz), 50.1, 44.8, 44.3 (minor), 43.2 (minor), 42.4, 41.6 (minor), 41.4, 39.6, 38.5 (minor), 36.9.

The crystalline free base can also be isolated as follows:
(a) The reaction mixture upon completion of the hydrogenation step is charged with 25 wt % of Ecosorb C-941. The mixture is stirred under nitrogen for one h and then filtered. The cake is washed with 2 L/kg of methanol. Recovery of free base is about 95% and optical purity about 95% ee.
(b) The freebase solution in methanol is concentrated to 3.5-4.0 L/kg volume (based on free base charge) and then solvent-switched into isopropanol (EPA) to final volume of 3.0 L/kg IPA.
(c) The slurry is heated to 40° C. and aged 1 h at 40° C. and then cooled to 25° C. over 2 h.
(d) Heptane (7 L/kg) is charged over 7 h and the slurry stirred for 12 h at 22-25° C. The supernatant concentration before filtering is 10-12 mg/g.
(e) The slurry is filtered and the solid washed with 30% IPA/heptane (2L/kg).
(f) The solid is dried in a vacuum oven at 40° C.
(g) The optical purity of the free base is about 99% ee.

The following high-performance liquid chromatographic (HPLC) conditions were used to determine percent conversion to product:
Column: Waters Symmetry C18, 250 mm×4.6 mm
Eluent: Solvent A: 0.1 vol % HClO₄/H₂O
   Solvent B: acetonitrile
Gradient: 0 min 75% A: 25% B
   10 min 25% A: 75% B
   12.5 min 25% A: 75% B
   15 min 75% A: 25% B
Flow rate: 1 mL/min
Injection Vol.: 10 μL
UV detection: 210 nm
Column temp.: 40° C.
Retention times: compound 2-4: 9.1 min
   compound 2-5: 5.4 min
   tBu Josiphos: 8.7 min

The following high-performance liquid chromatographic (HPLC) conditions were used to determine optical purity:
Column: Chirapak, AD-H, 250 mm×4.6 mm
Eluent: Solvent A: 0.2 vol. % diethylanine in heptane
   Solvent B: 0.1 vol % diethylamine in ethanol
Isochratic Run Time: 18 min
Flow rate: 0.7 mL/min
Injection Vol.: 7 μL
UV detection: 268 nm
Column temp.: 35° C.
Retention times: (R)-amine 2-5: 13.8 min
   (S)-amine 2-5: 11.2 min

The following non-limiting Examples are intended to illustrate the present invention and should not be construed as being limitations on the scope or spirit of the instant invention.

### EXAMPLE 1

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine hydrochloric acid salt monohydrate

Compound I freebase (20 mg) was dissolved in 0.25 ml of 90% isopropanol/methanol (v/v). A solution of HCl in diethyl ether (0.025 ml, 2 M solution) was added. A thick slurry of crystals formed. The mixture was heated to 55° C. and then slowly cooled to room temperature. The solid was filtered and washed with IPA. The crystal form of the solids was shown to be a monohydrate by the physical methods below.

### EXAMPLE 2

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine L-tartaric acid salt hemihydrate

Compound I free base (1.80 g) was dissolved in 90 mL of IPA and heated to 50° C. A solution of L-tartaric acid in water (0.675 g in 9 mL water) was added. A thick slurry formed which was heated to 60° C. and aged overnight (about 18 h). The solution was filtered and washed with IPA and then dried in a vacuum oven at 40° C. with a nitrogen sweep. The crystal form of the solids was shown to be a hemihydrate by the physical methods below.

### EXAMPLE 3

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine benzenesulfonic acid salt anhydrate

Compound I free base (10.40 g) was dissolved in 520 mL of isopropyl acetate (IPAc). The solution was heated to 50° C. and a solution of benzenesulfonic acid (4.10 g) in 50 mL IPAc was added to the solution over one h. After 20% of the addition, the solution was seeded with 0.1% benzenesulfonic acid salt and the addition was resumed. Upon complete addition, the slurry was cooled to room temperature and then filtered and washed with 25 mL of IPA and 50 mL of hexanes. The solids were dried on the filter frit with a nitrogen sweep. The crystal form of the solids was shown to be an anhydrate by the physical methods below.

### EXAMPLE 4

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine Q-toluenesulfonic acid salt anhydrate

1.15 g of p-Toluenesulfonic acid in methanol (5 mL) was added to 5.25 g of a 47 wt % solution of Compound I free base in methanol. A slurry formed and the mixture was charged with 15 mL methyl-tert-butyl ether (MTBE). The slurry was filtered and then washed with 5 mL of MTBE. The solids were dried on the frit. The crystal form of the solids was shown to be an anhydrate by the physical methods below.

### EXAMPLE 5

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine (1S)-(+)-camphorsulfonic acid salt anhydrate

A solution of Compound I free base in 3L/kg of methanol was charged with 1.0 equivalent of (1S)-(+)-camphorsulfonic acid. The solution was aged and a slurry developed. 7 L/kg of MTBE was added to the slurry and the mixture was aged at room temperature. The slurry was filtered and then washed with MTBE. The solids were dried at 40° C. in a vacuum oven under a nitrogen gas sweep. The crystal form of the solids was shown to be an anhydrate by the physical methods below.

X-ray powder diffraction studies are widely used to characterize crystalline structures, crystallinity, and polymorphism. The X-ray powder diffraction patterns of the various crystalline salts of the present invention were generated on a Philips Analytical X'Pert PRO X-ray Diffraction System with PW3040/60 console. A PW3373/00 ceramic Cu LEF X-ray tube K-Alpha radiation was used as the source.

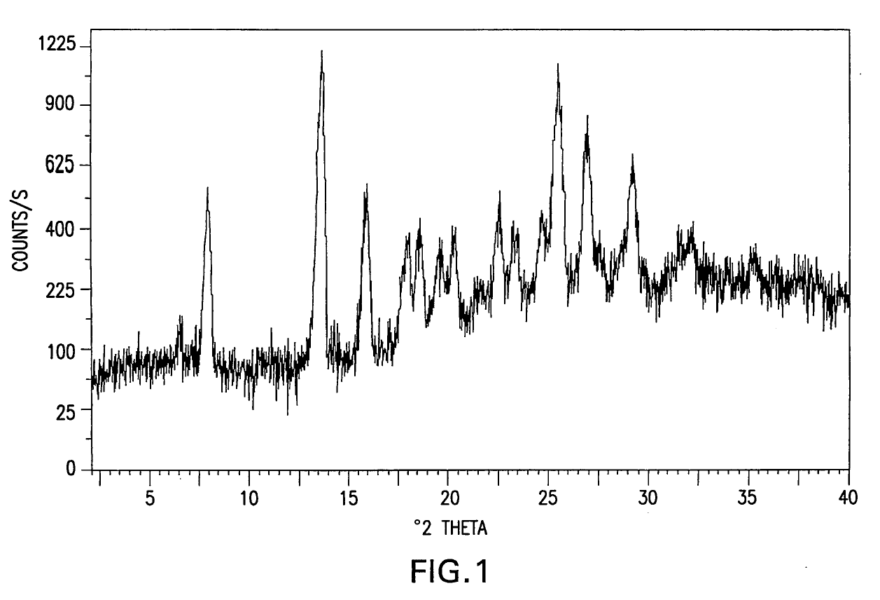

FIG. 1 shows the X-ray diffraction pattern for the crystalline hydrochloric acid salt monohydrate of Compound I of the present invention. The hydrochloric acid salt exhibited characteristic diffraction peaks corresponding to d-spacings of 3.0, 3.3, 3.5, 6.5, and 11.0 angstroms.

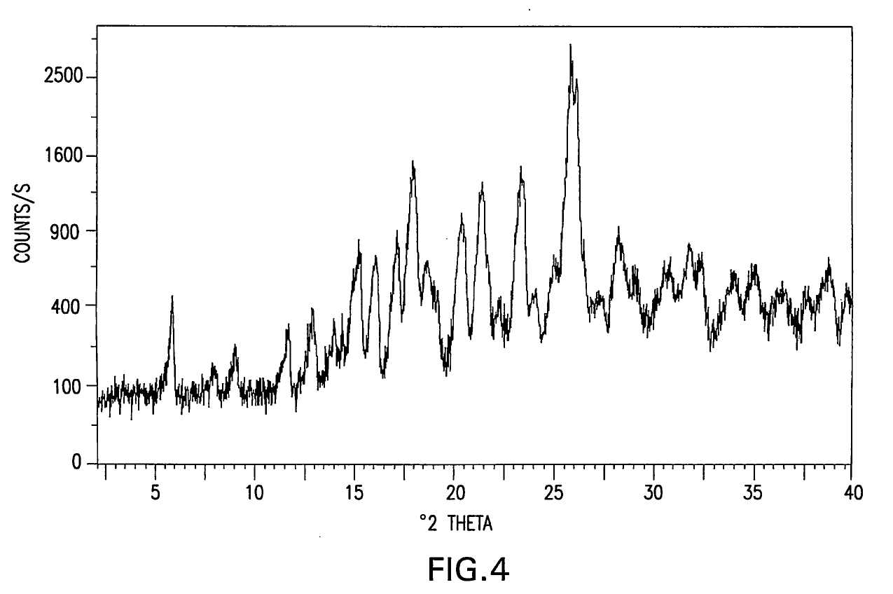

FIG. 4 shows the X-ray diffraction pattern for the crystalline L-tartaric acid salt hemihydrate of Compound I of the present invention. The L-tartaric acid salt exhibited characteristic diffraction peaks corresponding to d-spacings of 3.2, 3.4, 3.8, 4.1, 4.3, 4.9, and 5.8 angstroms.

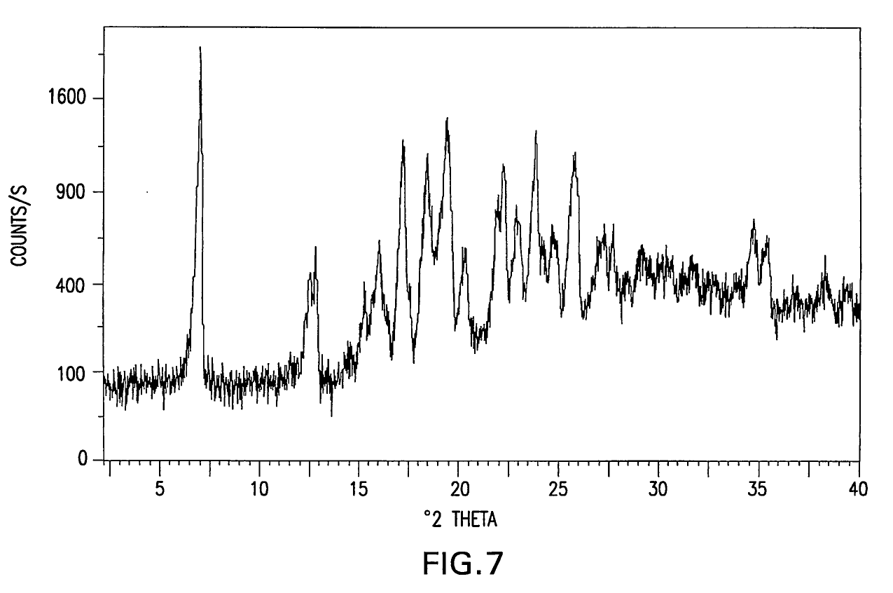

FIG. 7 shows the X-ray diffraction pattern for the crystalline benzenesulfonic acid salt anhydrate of Compound I of the present invention. The benzenesulfonic acid salt exhibited characteristic diffraction peaks corresponding to d-spacings of 3.4, 3.7, 4.0, 4.6, 4.8, 5.2, and 12.7 angstroms.

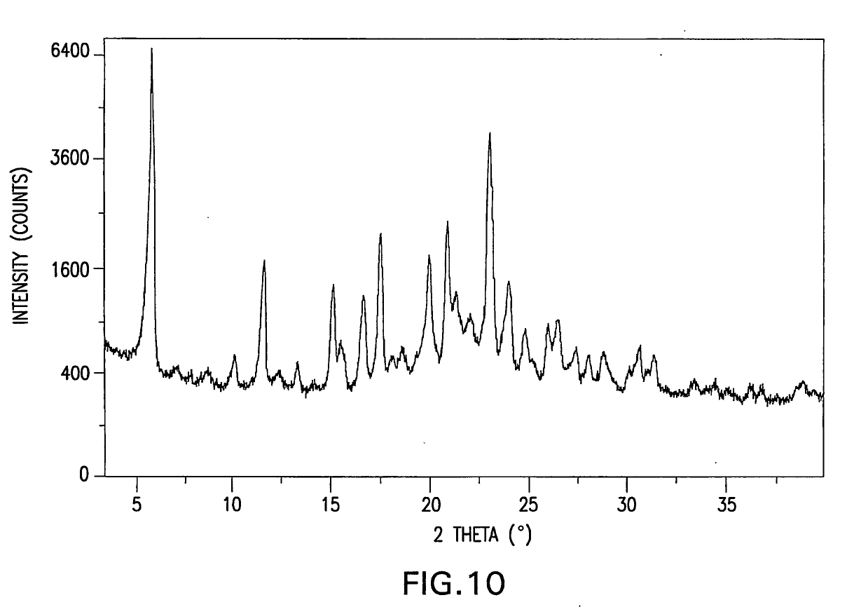

FIG. 10 shows the X-ray diffraction pattern for the crystalline p-toluenesulfonic acid salt anhydrate of Compound I of the present invention. The p-toluenesulfonic acid salt exhibited characteristic diffraction peaks corresponding to d-spacings of 3.9, 4.3, 4.5, 5.1, 5.7, 5.9, 7.6, and 15.0 angstroms.

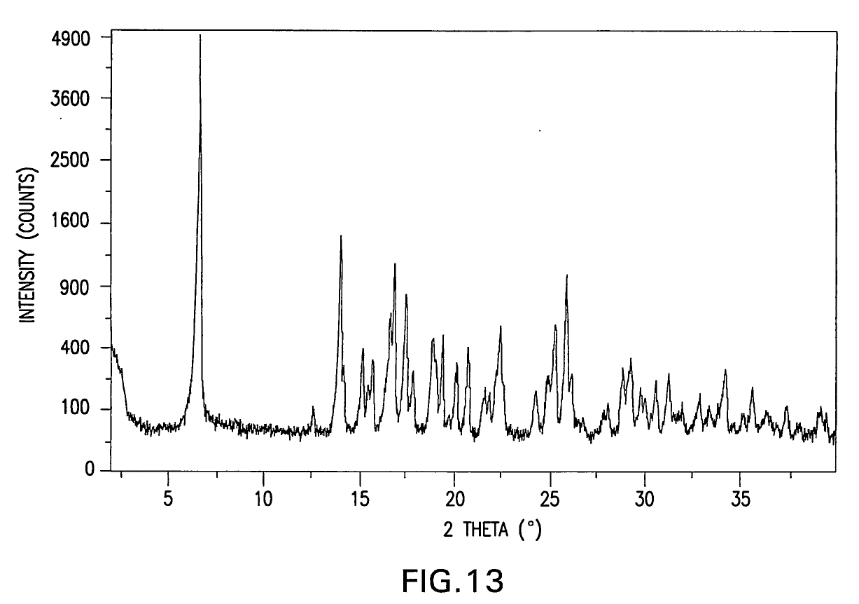

FIG. 13 shows the X-ray diffraction pattern for the crystalline (1S)-(+)-10-camphorsulfonic acid salt anhydrate of Compound I of the present invention. The 10-camphorsulfonic acid salt exhibited characteristic diffraction peaks corresponding to d-spacings of 3.4, 3.5, 4.0, 5.1, 5.3, 6.3, and 13.5 angstroms.

In addition to the X-ray powder diffraction patterns described above, the crystalline salts of Compound I of the present invention were further characterized by means of their differential scanning calorimetry (DSC) curves and their thermogravimetric analysis (TGA) curves.

A TA Instruments DSC 2910 or equivalent instrumentation was used to obtain the DSC curves. Between 2 and 6 mg sample was weighed into an open pan. This pan was then crimped and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature, and enthalpy.

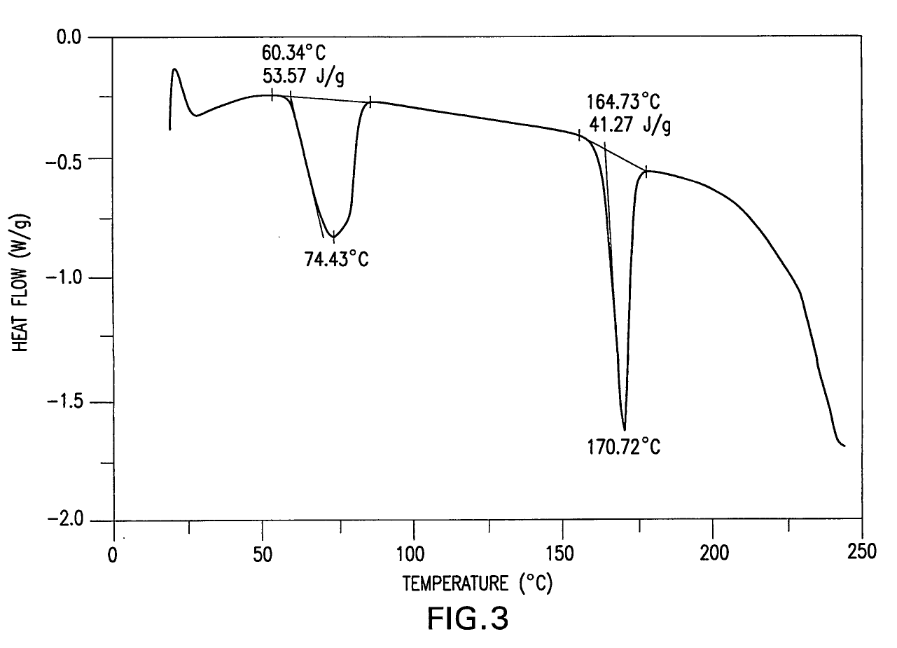

FIG. 3 shows a characteristic DSC curve for the crystalline hydrochloric acid salt monohydrate of Compound I. The hydrochloric acid salt exhibited a broad endotherm at about 74° C., attributed to evolution of water, with an onset temperature of about 60° C. and an enthalpy of about 54 J/g and a melting endotherm with an onset temperature of about 165° C., a peak temperature of about 170° C., and an enthalpy of about 41 J/g.

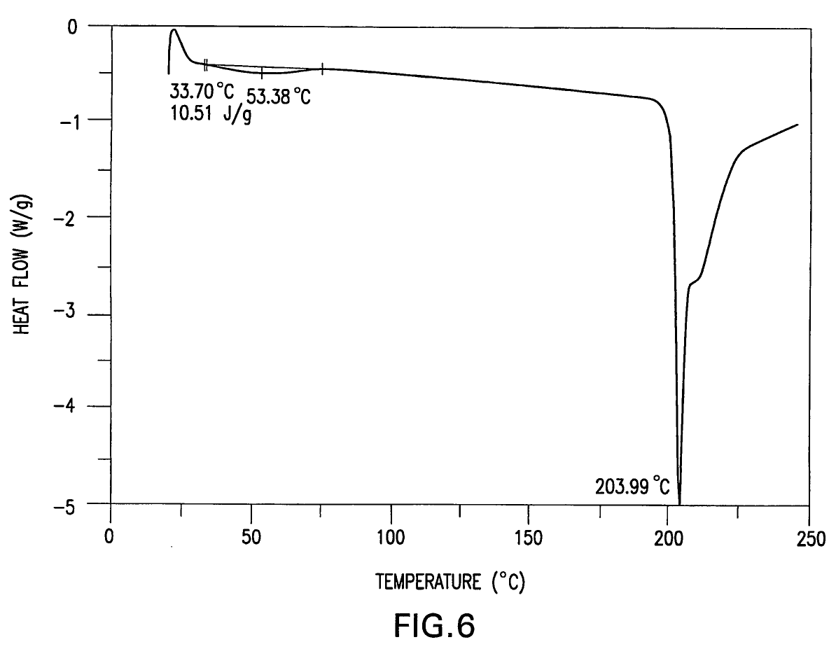

FIG. 6 shows a characteristic DSC curve for the crystalline L-tartaric acid salt hemihydrate of Compound I. The L-tartaric acid salt exhibited a broad endotherm at about 54° C., attributed to evolution of water, with an onset temperature of about 34° C. and an enthalpy of about 11 J/g and a melting and decomposition endotherm with a peak temperature of about 204° C.

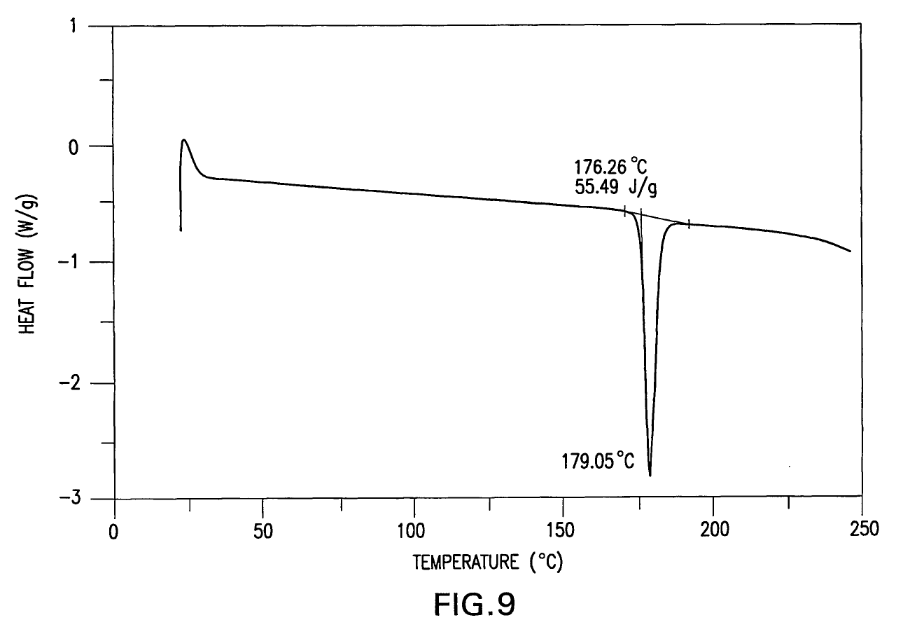

FIG. 9 shows a characteristic DSC curve for the crystalline benzenesulfonic acid salt anhydrate of Compound I. The benzenesulfonic acid salt exhibited a sharp melting endotherm with an onset temperature of about 176° C., a peak temperature of about 179° C., and an enthalpy of about 55 J/g.

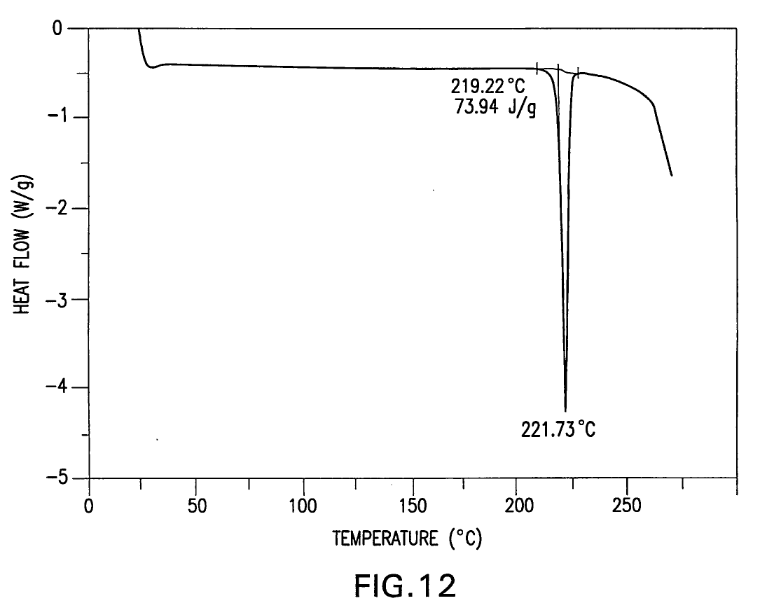

FIG. 12 shows a characteristic DSC curve for the crystalline p-toluenesulfonic acid salt anhydrate of Compound I. The p-toluenesulfonic acid salt exhibited a sharp melting endotherm with an onset temperature of about 219° C., a peak temperature of about 222° C., and an enthalpy of about 74 J/g.

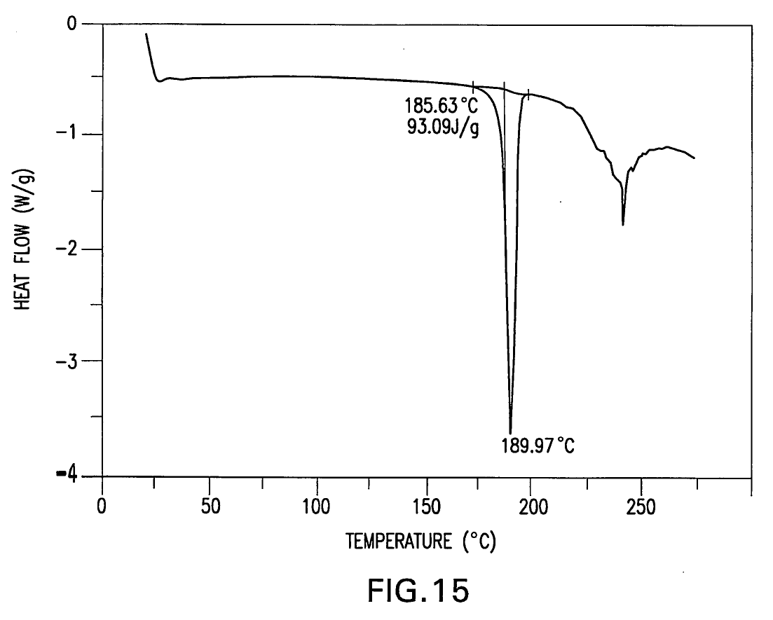

FIG. 15 shows a characteristic DSC curve for the crystalline (1S)-(+)-10-camphorsulfonic acid salt anhydrate of Compound I. The camphorsulfonic acid salt anhydrate exhibited a sharp melting endotherm with an onset temperature of about 186° C., a peak temperature of about 190° C., and an enthalpy of about 93 J/g.

A Perkin Elmer model TGA 7 or equivalent instrument was used to obtain the TGA curves. Experiments were performed under a flow of nitrogen and using a heating rate of 10° C./min to a maximum temperature of approximately 250° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss was to be calculated. Weight losses are reported up to the onset of decomposition/evaporation.

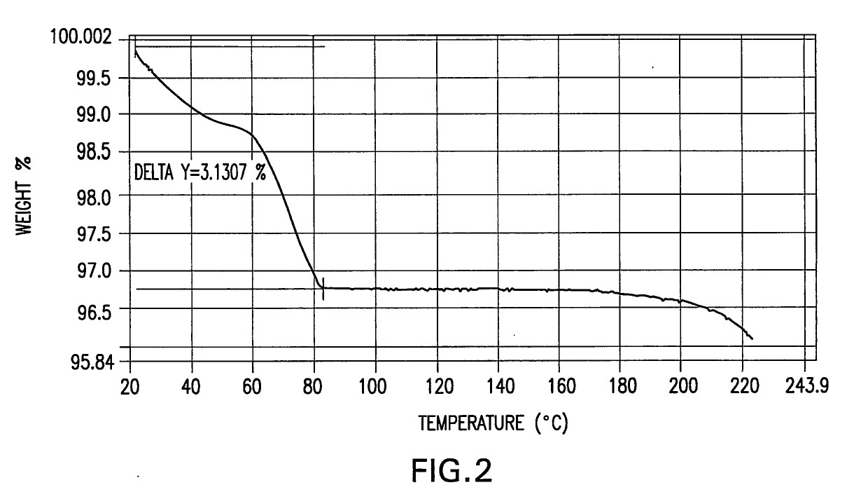

FIG. 2 shows a characteristic thermogravimetric analysis (TGA) curve for the crystalline hydrochloric acid salt monohydrate of Compound I. TGA indicated a weight loss of about 3.1% from ambient temperature to about 83° C.

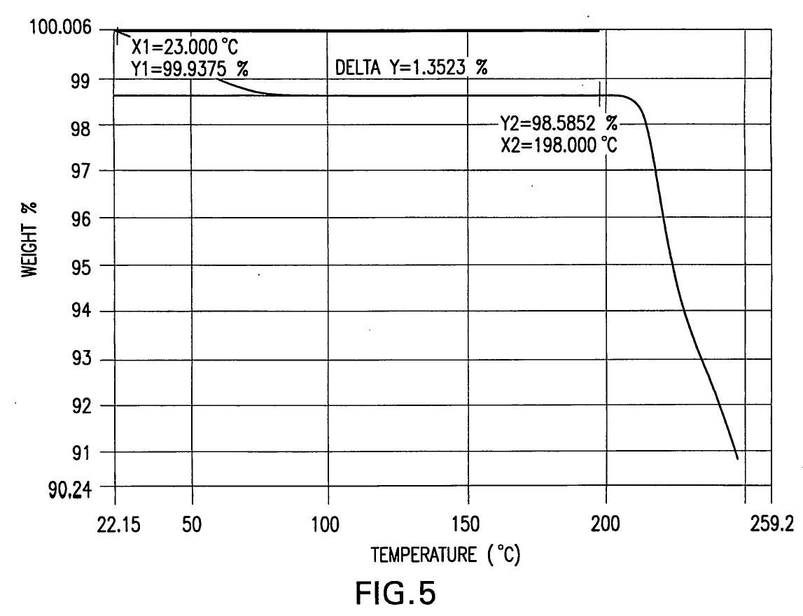

FIG. 5 shows a characteristic thermogravimetric analysis (TGA) curve for the crystalline L-tartaric acid salt hemihydrate of Compound I. TGA indicated a weight loss of about 1.4% from ambient temperature to about 198° C.

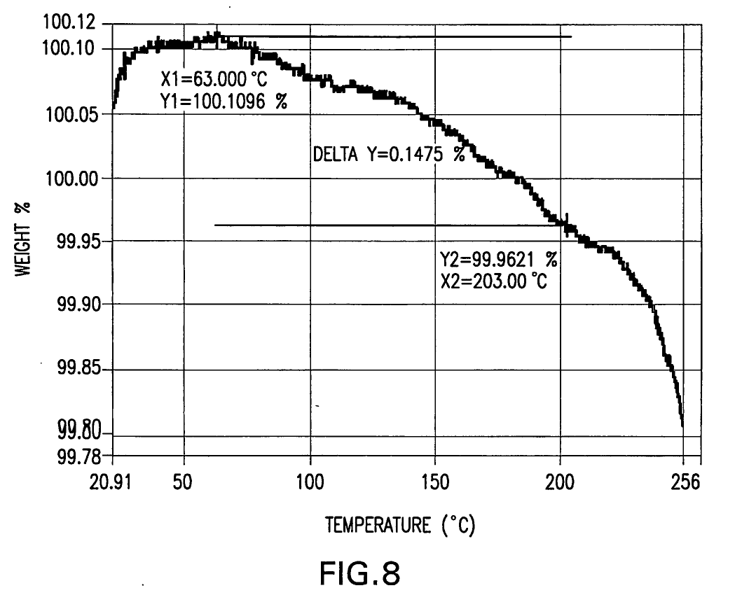

FIG. 8 shows a characteristic thermogravimetric analysis (TGA) curve for the crystalline benzenesulfonic acid salt anhydrate of Compound I. TGA indicated a weight loss of about 0.1% from about 63° C. to about 203° C.

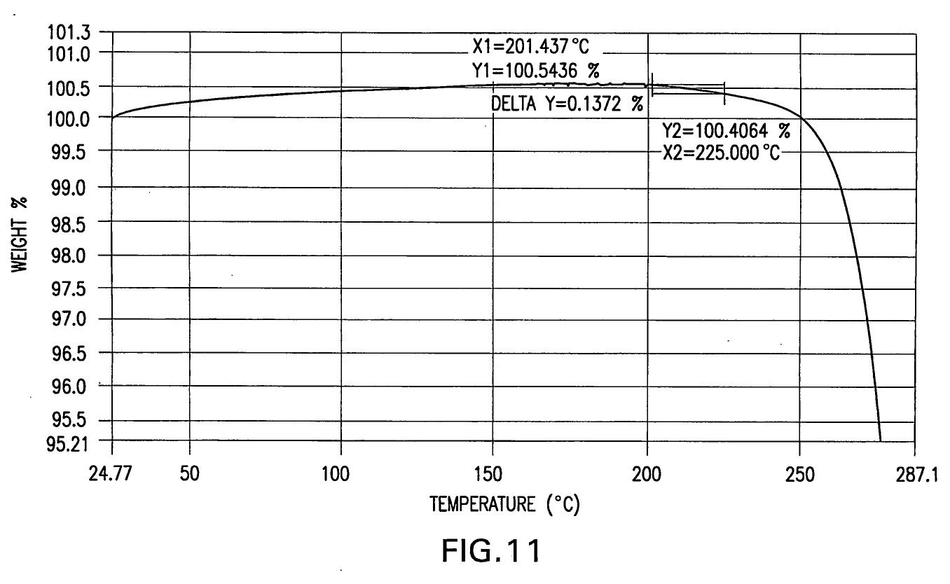

FIG. 11 shows a characteristic thermogravimetric analysis (TGA) curve for the crystalline p-toluenesulfonic acid salt anhydrate of Compound I. TGA indicated a weight loss of about 0.1% from ambient temperature to about 225° C.

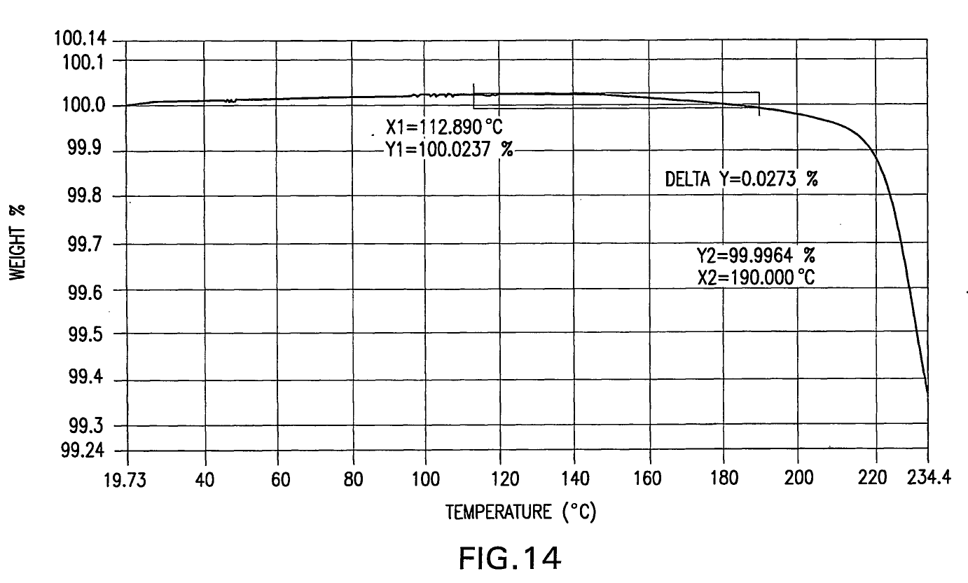

FIG. 14 shows a characteristic thermogravimetric analysis (TGA) curve for the crystalline (1S)-(+)-10-camphorsulfonic acid salt anhydrate of Compound I. TGA indicated a weight loss of about 0.0% from ambient temperature to about 190° C.

The crystalline salts of the present invention have a phase purity of at least about 5% of the form with the above X-ray powder diffraction and DSC physical characteristics. In one embodiment the phase purity is at least about 10% of the form with the above solid-state physical characteristics. In a second embodiment the phase purity is at least about 25% of the form with the above solid-state physical characteristics. In a third embodiment the phase purity is at least about 50% of the form with the above solid-state physical characteristics. In a fourth embodiment the phase purity is at least about 75% of the form with the above solid-state physical characteristics. In a fifth embodiment the phase purity is at least about 90% of the form with the above solid-state physical characteristics. In a sixth embodiment the crystalline salts of the present invention are the substantially phase pure forms with the above solid-state physical characteristics. By the term “phase purity” is meant the solid state purity of the particular salt with regard to a particular crystalline form of the salt as determined by the solid-state physical methods described in the present application.

### Examples of Pharmaceutical Compositions:

The crystalline salts of the present invention can be formulated into a tablet by a direct compression process. A 100 mg potency tablet is composed of 100 mg of the active ingredient, 276 mg mannitol, 20 mg of croscarmellose sodium, and 4 mg of magnesium stearate. The active ingredient, microcrystalline cellulose, and croscarmellose are first blended, and the mixture is then lubricated with magnesium stearate and pressed into tablets.

1. A crystalline salt of (2R)-4-oxo-4-[3-(trifluoromethyl)-5,6-dihydro[1,2,4]triazolo[4,3-α]pyrazin-7(8h)-yl]-1-(2,4,5-trifluorophenyl)butan-2-amine of structural formula I: 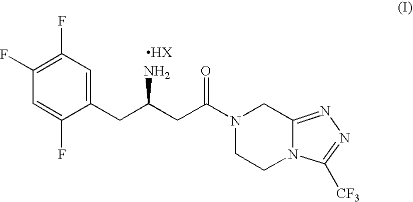
or a hydrate thereof;
wherein HX is an acid selected from the group consisting of hydrochloric acid, tartaric acid, benzenesulfonic acid, p-toluenesulfonic acid, and 10-camphorsulfonic acid.

2. The crystalline salt of claim 1 wherein said acid is hydrochloric acid.

3. The crystalline salt of claim 1 wherein said acid is benzenesulfonic acid.

4. The crystalline salt of claim 1 wherein said acid is p-toluenesulfonic acid.

5. The crystalline salt of claim 1 wherein said acid is tartaric acid.

6. The crystalline salt of claim 1 wherein said acid is 10-camphorsulfonic acid.

7. The crystalline salt of claim 5 wherein said tartaric acid is L-tartaric acid.

8. The crystalline salt of claim 5 wherein said tartaric acid is D-tartaric acid.

9. The crystalline salt of claim 6 wherein said 10-camphorsulfonic acid is (1S)-(+)-10-camphorsulfonic acid.

10. The crystalline salt of claim 6 wherein said 10-camphorsulfonic acid is (1R)-(−)-10-camphorsulfonic acid.

11. The crystalline hydrochloric acid salt of claim 2 characterized as being a monohydrate.

12. The crystalline hydrochloric acid salt of claim 11 characterized by characteristic reflections obtained from the X-ray powder diffraction pattern at spectral d-spacings of 3.0, 3.3, 3.5, 6.5, and 11.0 angstroms.

13. The crystalline hydrochloric acid salt of claim 12 further characterized by the X-ray powder diffraction pattern of FIG. 1.

14. The crystalline hydrochloric acid salt of claim 11 further characterized by the differential scanning calorimetric (DSC) curve of FIG. 3.

15. The crystalline hydrochloric acid salt of claim 11 further characterized by the thermogravimetric analysis (TGA) curve of FIG. 2.

16. The crystalline L-tartaric acid salt of claim 7 characterized as being a hemihydrate.

17. The crystalline L-tartaric acid salt of claim 16 characterized by characteristic reflections obtained from the X-ray powder diffraction pattern at spectral d-spacings of 3.2, 3.4, 3.8, 4.1, 4.3, 4.9, and 5.8 angstroms.

18. The crystalline L-tartaric acid salt of claim 17 further characterized by the X-ray powder diffraction pattern of FIG. 4.

19. The crystalline L-tartaric acid salt of claim 16 further characterized by the differential scanning calorimetric (DSC) curve of FIG. 6.

20. The crystalline L-tartaric acid salt of claim 16 further characterized by the thermogravimetric analysis (TGA) curve of FIG. 5.

21. The crystalline benzenesulfonic acid of claim 3 characterized as being an anhydrate.

22. The crystalline benzenesulfonic acid salt of claim 21 characterized by characteristic reflections obtained from the X-ray powder diffraction pattern at spectral d-spacings of 3.4, 3.7, 4.0, 4.6, 4.8, 5.2, and 12.7 angstroms.

23. The crystalline benzenesulfonic acid salt of claim 22 further characterized by the X-ray powder diffraction pattern of FIG. 7.

24. The crystalline benzenesulfonic acid salt of claim 21 further characterized by the differential scanning calorimetric (DSC) curve of FIG. 9.

25. The crystalline benzenesulfonic acid salt of claim 21 further characterized by the thermogravimetric analysis (TGA) curve of FIG. 8.

26. The crystalline p-toluenesulfonic acid salt of claim 4 characterized as being an anhydrate.

27. The crystalline p-toluenesulfonic acid salt of claim 26 characterized by characteristic reflections obtained from the X-ray powder diffraction pattern at spectral d-spacings of 3.9, 4.3, 4.5, 5.1, 5.7, 5.9, 7.6, and 15.0 angstroms.

28. The crystalline p-toluenesulfonic acid salt of claim 27 further characterized by the X-ray powder diffraction pattern of FIG. 10.

29. The crystalline p-toluenesulfonic acid salt of claim 26 further characterized by the differential scanning calorimetric (DSC) curve of FIG. 12.

30. The crystalline p-toluenesulfonic acid salt of claim 26 further characterized by the thermogravimetric analysis (TGA) curve of FIG. 11.

31. The crystalline (1S)-(+)-10-camphorsulfonic acid salt of claim 9 characterized in being an anhydrate.

32. The crystalline (1S)-(+)-10-camphorsulfonic acid salt of claim 31 characterized by characteristic reflections obtained from the X-ray powder diffraction pattern at spectral d-spacings of 3.4, 3.5, 4.0, 5.1, 5.3, 6.3, and 13.5 angstroms.

33. The crystalline (1S)-(+)-10-camphorsulfonic acid salt of claim 32 further characterized by the X-ray powder diffraction pattern of FIG. 13.

34. The crystalline (1S)-(+)-10-camphorsulfonic acid salt of claim 31 further characterized by the differential scanning calorimetric (DSC) curve of FIG. 15.

35. The crystalline (1S)-(+)-10-camphorsulfonic acid salt of claim 31 further characterized by the thermogravimetric analysis (TGA) curve of FIG. 14.

36. A pharmaceutical composition comprising a therapeutically effective amount of a salt according to claim 1 in association with one or more pharmaceutically acceptable carriers or excipients.

37. A method of treating Type 2 diabetes comprising administering to a mammal in need of such treatment a therapeutically effective amount of a salt according to claim 1.

38. (canceled)
